(19) United States
(12) Patent Application Publication
(10) Pub. No.: US 2019/0328314 A1
(43) Pub. Date: Oct. 31, 2019

(54) METHOD AND APPARATUS FOR MONITORING AND IMPROVING LIFESTYLE VIA A WEARABLE

(71) Applicant: Opter Life, Inc., Los Gatos, CA (US)

(72) Inventors: Cutter Coryell, Los Gatos, CA (US); Michael Coryell, Pleasant Hill, CA (US); Mark Wiprud, Austin, TX (US); Chalisa Prarasri, Los Gatos, CA (US); Admiral Potato, Salt Lake City, UT (US)

(21) Appl. No.: 16/396,625

(22) Filed: Apr. 26, 2019

Related U.S. Application Data
(60) Provisional application No. 62/663,227, filed on Apr. 26, 2018.

(51) Int. Cl. A61B 5/00; A61B 5/11

(52) U.S. Cl. CPC A61B 5/4561; A61B 5/1116; A61B 5/6822; A61B 5/6814; A61B 5/0002

(57) ABSTRACT

One embodiment can include a computing device with a display, a wireless transceiver, and a processor. The wireless transceiver can be configured to receive data regarding sets of values from a wearable posture device of a user. Each set can have three values representing the three orthogonal acceleration values of an accelerometer in the wearable posture device. The three orthogonal acceleration values can be used to represent a posture of the user. The processor can be configured to operate on the received data to generate visualizations tracking the postures of the user. The display can be configured to present the visualizations generated. In one embodiment, the visualizations could be 3D. In another embodiment, the visualizations could be two 2D images, one representing front views and the other side views of the user.

### CROSS REFERENCE TO RELATED APPLICATION

This application claims benefit of U.S. Provisional Patent Application No. 62/663,227, filed Apr. 26, 2018, and entitled “METHOD AND APPARATUS FOR MONITORING AND IMPROVING LIFESTYLE VIA A WEARABLE,” which is hereby incorporated herein by reference.

### BACKGROUND OF THE INVENTION

Good postures are essential to our health and well-being.

If we are standing, sitting or lying down, gravity pulls our joints, ligaments and muscles down. Good postures distribute the pull across our body so as not to overstress any part of it. Good postures also reduce fatigue and strain on our body, particularly our spine. If we are exercising or even just walking, good postures help keep our balance, resulting to fewer injuries. On the other hand, poor postures could weaken our core muscles, which further encourage slumping, deteriorating our postures.

The proliferations of computers and smartphones have exacerbated poor postures. Everywhere we could see people slouched, with their heads tilted down, totally engrossed in their screens, for long durations of time.

The first step to break the habits of poor postures is to be aware of them.

It should be apparent from the foregoing that there is a need to alert many of their poor postures, and help them improve.

### SUMMARY OF THE INVENTION

The invention can be implemented in numerous ways including methods, systems, devices, and computer readable media. Several embodiments of the invention are discussed below.

In one embodiment, a wearable apparatus can at least monitor the posture of a user. The apparatus can include an accelerometer and a wireless transceiver. At preset intervals, the apparatus could extract sets of three values from the accelerometer, representing three orthogonal values of an acceleration vector. Each set provides information regarding a posture of the user.

The wireless transceiver can transmit data regarding the sets of values from the accelerometer to a computing device of a user, such as the user's cell phone. The mobile device includes an app to analyze the sets of values to generate visualizations tracking the postures of the user. The visualizations could be presented by a display of the computing device.

In another embodiment, the posture device could be performing at least some of the analysis, and the wireless transceiver could transmit the analysis results to the computing device to present the visualizations by the display of the computing device.

As the user changes her posture, the visualizations can follow. In one embodiment, the visualizations could be in 3 dimensions.

In one embodiment, the 3D visualizations could be represented in two 2D images, one as side views of the user, and the other as front views of the user.

In one embodiment, the app could also alert the user to adjust the user's posture if necessary.

In one embodiment, the posture-monitoring wearable apparatus can be in the format of a pendant.

Other aspects and advantages of the present invention will become apparent from the following detailed description, which, when taken in conjunction with the accompanying drawings, illustrates by way of example the principles of the invention.

### BRIEF DESCRIPTION OF THE DRAWINGS

FIG. 1 shows an embodiment of a posture device in a configuration of a pendant that could be worn via a chain.

FIG. 2 shows an embodiment regarding posture visualization.

FIG. 3 shows embodiments of a posture device and a clip.

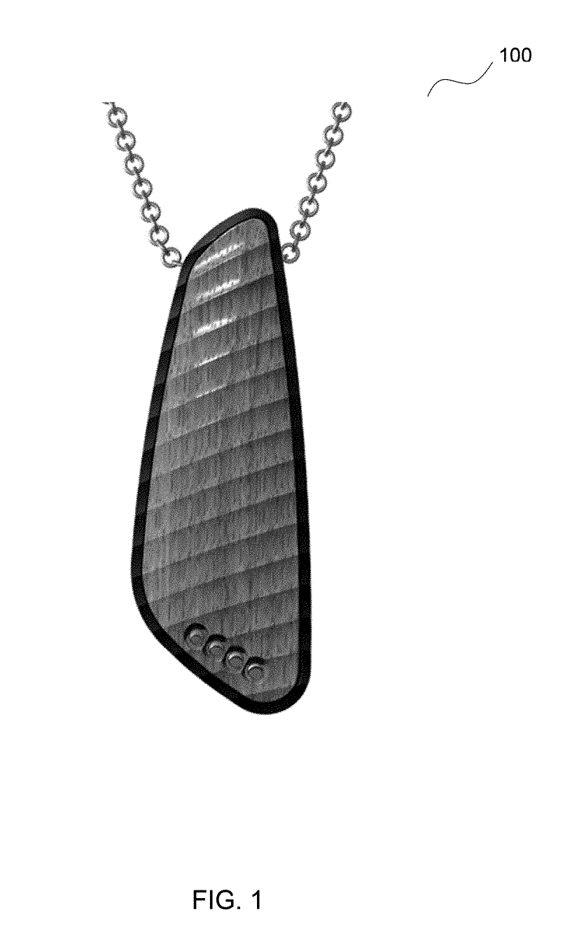

Same numerals in FIGS. 1-3 are assigned to similar elements in all the figures. Embodiments of the invention are discussed below with reference to FIGS. 1-3. However, those skilled in the art will readily appreciate that the detailed description given herein with respect to these figures is for explanatory purposes as the invention extends beyond these limited embodiments.

### DETAILED DESCRIPTION OF THE INVENTION

A number of embodiments of the invention include a lifestyle-monitoring wearable apparatus configured to monitor one or more attributes of a user's lifestyle, and/or to improve one or more attributes of the user's lifestyle. The attributes could include, for example, posture, exposure to UV, putting on sunscreen, exposure to visible light, sleep, steps taken, exercise, and calories consumed. The apparatus can be wirelessly coupled to a computing device, which can be a mobile device (e.g., a user's cell phone). The computing device could include an application program (app) to, for example, graphically display the monitored attributes and/or to help lifestyle improvements.

In one embodiment, the lifestyle-monitoring-wearable apparatus includes a radiation-monitoring module. With the module, the apparatus could, for example, help prevent the user from getting too much UV, and/or improve the sleep health of the user. To illustrate, for proper sleep health, one may want bright light in the late morning to early afternoon, and dim light in the early morning and evening.

In one embodiment, the radiation-monitoring module can include one or more radiation detectors. The detectors could be coupled to one or more radiation guides or channels.

One radiation detector can be a UV detector. The UV detector should face the sun for maximum detection. One way to measure UV can be by measuring UV directly via the UV detector. Another way can be by calculating the UV index based on a ratio of measurements, such as based on outputs from the UV detector and a blue light detector. A third way can be based on downloading the local UV index from EPA in the area the user is at. The different ways could be used together.

The radiation-monitoring module can include a blue light detector to detect blue light, which is thought to inhibit melatonin production. This can be used to enhance sleep health. In one approach, the apparatus could position the blue light detector so that the detector can be orientated to approximately point directly in front of the user when the apparatus is being worn in its usual position.

The radiation-monitoring module can also include other radiation detectors, such as a green light detector.

In one approach, a radiation detector can include a LED electrically connected in parallel with a capacitor. To detect different types of radiation, different LEDs can be selected that are sensitive to the different radiation ranges, such as UV/green/blue ranges. After selection, the responses of the LEDs can be calibrated, such as by charting their input currents versus their output illumination levels.

LEDs also can operate to generate small amount of currents when they receive photons of certain wavelengths. The small amount of currents could build up charges on the corresponding capacitors. For each detector, periodically, the corresponding capacitor can be shorted to ground. Then the capacitor again collects charges from the current of the corresponding LED to build up its voltage. In one approach, when the voltage across the capacitor rises to a certain threshold, a low-power comparator triggers a CPU interrupt. The time it takes to reach the threshold can thus be monitored. The apparatus can use firmware to map the monitored time to reach the threshold into an estimated illumination level.

In one embodiment, the radiation-monitoring module can include one or more radiation or light guide to guide the different incident radiations to their corresponding detectors. For example, each detector could have its own radiation guide (e.g. a fiber optic tube), or one radiation guide (e.g. a plastic block) could be used for the different detectors.

The radiation-monitoring module could include a number of protrusions, protruding a small amount (e.g. 1 mm) from the surface of the lifestyle-monitoring-wearable apparatus to enhance capturing the different radiations. The tips of the protrusions can be sanded to reduce sharpness and augment radiation coupling.

The lifestyle-monitoring-wearable apparatus could include a charging light indicator. The indicator can be built into the radiation-monitoring module, with its own radiation guide, or can share the one radiation guide of the detectors. In one embodiment, during charging, the apparatus could turn on the charging light indicator, and could stop analyzing data from the detectors.

One embodiment of the lifestyle-monitoring-wearable apparatus can be configured to operate as a posture device for a user's postures. The device can monitor and/or improve the postures of the user, using, for example, an accelerometer. The device could include a processor to extract raw outputs from the accelerometer at regular or preset intervals. The raw outputs can be known as acceleration vectors. As an example, if the user freely hangs the device in front of herself, the acceleration vector could measure the gravity pull. The gravity pull can be called the gravity vector. The gravity vector can be represented by three orthogonal values, the x, y, z values, of the gravity pull. Continuing on with the example of the free hanging device in front of the user, and with, for example, the device having radiation detectors facing away from her, the y value could represent the value along the axis pointing up/down, the z value as the one pointing perpendicular to the chest of the user (or as an example, parallel to a fiber optic tube, as one described herein), and x value as the value along the axis orthogonal to both the y and the z axes.

In one embodiment, the posture device can be configured as a pendant. FIG. 1 shows an example 100 of such an embodiment. The chain could be worn via a chain at, for example, the neck of a user.

Assume the user is wearing the pendant. To operate, in one embodiment, the user first calibrates the pendant by assuming good posture, and the user presses a “Calibrate Posture” virtual button via a user interface at an app, such as in a mobile device of the user, which could be the user's mobile phone. The posture device then extracts the accelerometer output, which can be a calibration vector, representing the vector along a calibration direction. This vector can represent a calibration pitch angle relative to the gravity vector (or the angle of rotation about the accelerometer's x axis). For example, the calibration pitch angle can be calculated as the arc tan(z value/y value).

After the calibration, the posture device could measure the user's postures. When measuring a user's posture, the accelerometer outputs can be the posture vector. In one approach, the posture vector can represent a posture pitch angle relative to the gravity vector. In one embodiment, the posture pitch angle can be compared to, or subtracted from, the calibration pitch angle of the user to identify the current posture of the user.

In one embodiment, the above calculations can be used to measure if the user has been slouching in the forward/backward orientation. In another embodiment, the posture device could be used to measure if the user has been slouching sideways, to check for spinal left/right alignment. The two embodiments could be similar, except that instead of measuring the pitch, the posture device could measure the roll, or the rotation about the z axis of the accelerometer. The roll angle can be calculated as the arc tan(y value/x value).

The posture device can keep taking posture vectors at a certain frequency, such as 25 Hz. The certain frequency could be a preset frequency or a variable frequency (or the frequency doesn't have to be fixed). Based on the different values from the posture vectors, the posture device could calculate and determine a moving average, such as an exponential moving average, of the posture pitch angle for a preset posture checking time period, which could be every minute.

In one embodiment, an exponential moving average of the posture pitch angle representing the user's posture can be categorized into different ranges, such as “good,” “mediocre,” “poor,” or “invalid.”

To illustrate, as an example, the ranges for the different moving averages could be:

Good posture: (calibration pitch angle−10°) to (calibration pitch angle+5°)

Mediocre posture: (calibration pitch angle+5°) to (calibration pitch angle+10°)

Bad posture: (calibration pitch angle+10°) to (calibration pitch angle+90°)

Invalid posture: (calibration pitch angle+90°) to (calibration pitch angle−10°)

In the above example, each of these ranges can be represented as a segment of the unit circle, together added up to the full 360°.

In one embodiment, for each posture checking time period, if no “good” postures were seen, and the number of “mediocre” postures added to the number of “poor” postures exceeds the number of “invalid” postures, the user's posture can require correction. This is one approach to determine if the user's posture requires correction.

In one embodiment, at every posture checking time period, the posture device could notify the user by, for example, using a vibration motor to vibrate the device if the user needs to correct her posture.

Periodically, the device could wirelessly (such as via Bluetooth) send data, such as data on raw vectors from the accelerometer, and/or data based on analysis on the raw vectors, to, for example, an app in the user's mobile phone. For example, if the app is opened, the accelerometer's raw vectors could be sent to the mobile phone, and could be used for visualization purposes. The period to transmit could be every minute, every 10 minutes, or at other intervals. The app could also show an alert message to notify the user if the user needs to correct her posture.

In one embodiment, an app in a mobile device, such as a mobile phone, could display a real-time, three-dimensional visualization of the user's posture. The algorithm for generating the posture visualization can be performed in the app, using, for example, streamed accelerometer data from, such as, a pendant as described herein.

In one approach, to generate the visualization, a user can first calibrate the pendant by assuming a good posture. Then the user could press a virtual button on the screen of the phone, which could be labelled “Calibrate Posture”. The most recent raw acceleration streamed from the pendant can be saved as the “calibration direction” at the time of calibration.

In one embodiment, the “calibration quaternion” Q can be defined to be the quaternion representing the rotation from the calibration direction to the vertical direction in user-coordinates.

In one embodiment, for each raw accelerometer sample A obtained from the pendant:
The “calibrated direction” can be calculated as QA/Q
The “posture rotation” R can be defined as the rotation from the calibrated direction to the vertical direction in user-coordinates
The “posture grade” can be defined to be the particular angle range (“good”, “mediocre”, “bad”, “invalid”, as described above) that the pitch of the calibrated direction falls into
Color could be used, with the color of the posture visualization set based on the posture grade to indicate severity of bad posture
The angle of rotation of each of the N invisible line segments that make up the spine (whose endpoints can be colored spheres in the visualization) can then be proportional to the angle of the rotation R, whereas the 3D axis of rotation of each segment can be the same as the axis of the rotation R.

On the screen of the display, visualizations related to the user's posture could appear. The visualization can show the user's body in side-profile and front-profile, and could have a cartoon head and spine indicating the direction the user is leaning. The cartoon head and spine can be rendered in 3D with perspective, so each of the two displays can present 3D information.

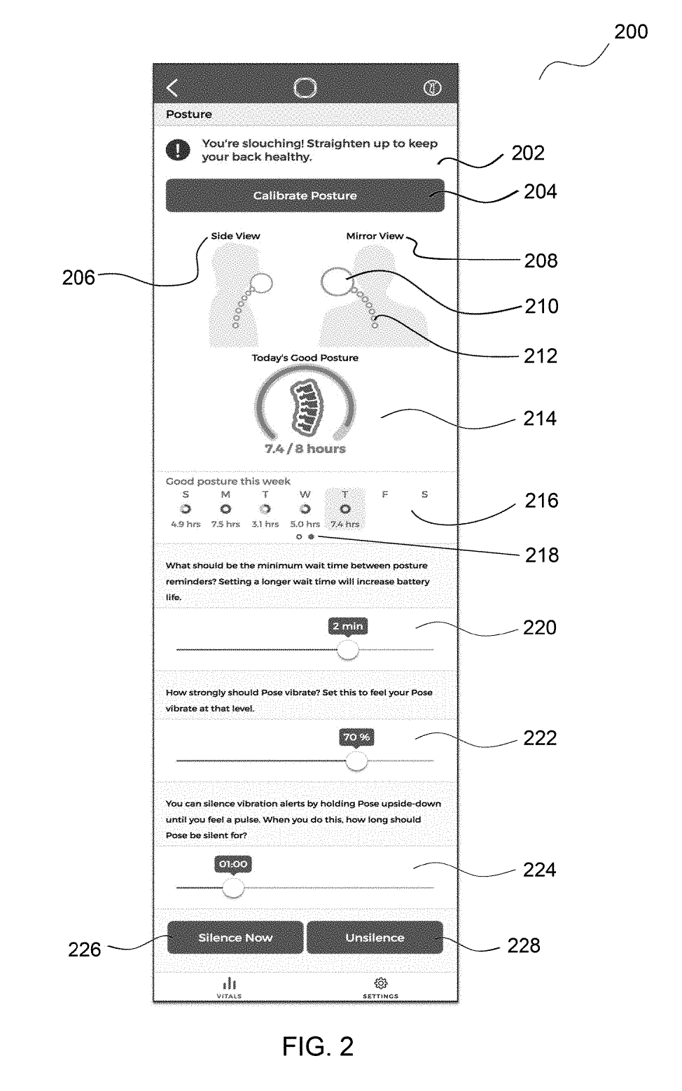

FIG. 2 shows an embodiment 200 regarding posture visualization. If the user's posture requires correction, the app can present a textual alert 202 at the top of the screen.

Beneath the textual alert (if present) can be the Calibrate Posture 204 virtual button. This button can be pressed to indicate that the user wishes to define the posture at that moment as good posture.

Beneath the Calibrate Posture button 204 can be a set of three-dimensional real-time visualizations of the user's body posture. One visualization 206 can depict the user from the side, and another 208 could depict the user as she would see herself in a mirror. To indicate the perspective of each visualization, there can be a silhouette of a person either in side-profile or front-profile.

Superimposed on the silhouettes can be cartoons of the user's head 210 and spine 212. Each cartoon can lean in a certain direction that can be the same as the direction the user is currently leaning, relative to the background silhouette. Additionally, the cartoons can possess three-dimensional perspective such that if the cartoon leans “towards the camera,” it could grow larger; and if the cartoon leans “away from the camera,” it could grow smaller. This 3D perspective can help the user understand which way the cartoon is leaning in three dimensions.

The cartoon and the silhouette can be colored according to the quality of the person's posture. For example, green can be good, yellow mediocre, and red poor posture.

Beneath the three-dimensional real-time posture visualizations can be a Today's Good Posture meter 214. The meter could show the duration of time the user has had “good” posture on that day relative to the user's goal. The coloration of the meter can depend on how “on-track” the user is for completing the user's goal before the end of the day.

Beneath the good posture meter 214 can be a display showing the amount of good posture for each day of that week 216. Like the Today's Good Posture meter, each day can possess a meter showing progress towards the goal, and a number showing how long the user had good posture for that day. This display can be swiped to show the statistics for the previous week. There could be two small dots 218 underneath indicating this functionality.

Beneath the weekly display of good posture can be a posture alert slider 220. This slider can allow the user to set the minimum delay between posture alerts. In one embodiment, the device can vibrate when the user's posture requires correction. This slider can modulate the maximum frequency of such posture alerts.

Beneath the posture alert delay slider 220 can be a vibration strength slider 222. This slider can allow the user to modify the strength at which the device vibrates during a posture alert, which can also affect the loudness. The posture device can vibrate as soon as the user changes the vibration strength level so the user can feel and/or hear the new strength.

Beneath the vibration strength slider 222 can be the silencing interval slider 224. This slider can allow the user to set the time interval for which the posture device should stop vibrating after a silencing gesture is performed. In one approach, the silencing gesture can be performed by holding the device upside-down for 5 to 15 seconds. After which the posture device could vibrate once to indicate that the silencing gesture has been recognized. Once the silencing interval has expired, the posture device can return to normal operating mode.

Beneath the silencing interval slider 224 can be two buttons. The first 226 can allow the user to initiate a silencing interval without using the silencing gesture described above, and the second 228 can allow the user to prematurely end a silencing interval and restore normal operating mode.

A number of embodiments described herein include coupling a posture device to a mobile device, such as the user's mobile phone, with the mobile device performing at least some of the operations regarding posture, such as to track, correct, and/or notify the user regarding the user's posture.

In one embodiment, a posture device can operate on its own without being coupled to another computing or mobile device. For example, the posture device could include an accelerometer, a physical calibration button, a processor, and an output generator. The output generator could be, for example, a vibration motor or a flashing light. The processor could receive data regarding sets of values, each set regarding three values representing three orthogonal acceleration values from the accelerometer, with the three orthogonal acceleration values being used to provide information regarding a posture of the user. The processor could also operate on the received data to analyze and track the postures of the user. Based on the analysis, the processor could notify the user by generating an output via the output generator to alert the user to improve on her posture, if the user's posture needs to be improved. To perform calibration, a user can push the button. In this embodiment, the posture device does not have to include a wireless transceiver. In another embodiment, a posture device doesn't include an external physical calibration button. A preset gesture could be used to initiate calibration. For example, one preset gesture can be holding the device upside-down (relative to how the device is normally used) for a certain period, such as 4 to 12 seconds. Such a gesture would initiate a calibration process. Note that one embodiment doesn't have to include an on/off button. For example, the device could be normally on, and if for 5 seconds, the range of posture vectors measured is below the noise level of the accelerometer, the device would be turned off. Then the device would be in a listen mode. When a posture vector has a value that is beyond a certain threshold, the device would be activated again.

In one embodiment, a lifestyle-monitoring wearable apparatus, which can be a posture device, doesn't include any physical buttons. The device can be controlled by gestures. This could make the device easier to manufacture and/or to waterproof. More than one gesture could be used to control the device. For example, assume that the device is paired to a user's first mobile phone. The device could be configured such that (a) if the user turns the device upside down for 5 to 15 seconds, the device could alert the user to start a calibration process. Instead of starting the calibration process, the user could couple this first gesture with a second gesture. For example, (b) the user turns the device right-side up (as the device is normally used) for such as, again 5 to 15 seconds, or till the device alerts the user with, for example, a flashing light. And if the user repeats steps (a) and (b) two times, at this point, the device would be configured to pair to a second mobile phone of the user; or the device could be configured to open a whitelist and allow a second mobile phone of the user to connect.

One embodiment for posture correcting and/or posture visualization can be performed in an app, which could be, for example, in a posture device, in a mobile device, such as a mobile phone, or a portion in a posture device and a portion in a mobile device. In one embodiment, a corresponding algorithm can be as follows:

To calibrate:
\vec{c}_{←}most recent 3D accelerometer measurement (the “calibration posture”)
R_{z}≡the rotation about the Z-axis by angle equal to atan 2(cₓ,c_{y})
c_{\vec{Y}Z}=R_{z}[\vec{c}]⇐\vec{c} rotated about the Z-axis until it lands in the YZ plane
Rₓ≡the rotation about the X-axis from (c_{\vec{Y}Z}) to the Y-axis. That could mean Rₓ[(c_{YZ})] pointing in the Y-direction. In summary, (RₓR_{z})[\vec{c}] pointing in the Y-direction.

To calculate a posture vector:
\vec{a}←the 3D accelerometer measurement.
\vec{d}=(RₓR_{z})[\vec{a}]⇐the “calibrated” posture vector.
θ=atan 2(a′_{z}, a′_{y})⇐the “pitch” of the user, i.e. forward rotation relative to calibration posture.
φ=atan 2(a′ₓ, a′_{y})⇐the “roll” of the user, i.e. leftward rotation relative to the calibration posture.
θ can then be histogrammed every “posture checking interval” to see what fraction of θs fall outside the “good” angle range, as described herein.
The same can be done for φ, vibrating if too many φs are outside an ideal angle range for roll during the posture checking interval.
Rₚₒₛₜᵤᵣₑ≡the rotation from the Y-axis to \vec{a}′ about ŷ×\vec{a}′, where y can be the unit vector in the Y-direction.
Rₚₒₛₜᵤᵣₑ can be transmitted to the app for the 3D visualization to use directly.

For the 3D visualization:
The 3D visualization code can receive Rₚₒₛₜᵤᵣₑ represented, for example, as a matrix, a quaternion, or an axis-angle pair.
At simplest, the 3D visualization could be an arrow in 3D space rotated from the Y-axis by Rₚₒₛₜᵤᵣₑ.

The posture visualization can be more complex than a simple 3D arrow, like a 3D human figure having a “spinal column” that deforms elastically. In that case, the rotation of each of N spinal segments can be given by Rₚₒₛₜᵤᵣₑ^{1/N}. Or, for example, a more realistic representation of the skeletal structure, such that Rₚₒₛₜᵤᵣₑ gives the rotation of the collarbone from its “good posture” orientation.

In one embodiment, based on, for example, the above algorithm, to calibrate, the user only requires to use one posture. Then to start calibration, the user can push a calibration button (which could be physical or virtual), or the user could perform a predetermined calibration gesture on the device, such as positioning the device a special way for a preset amount of time. In other words, calibration may not need multiple postures to complete.

In one approach, to calibrate, the user could place a posture device flat on the user's chest with the z-axis of the device substantially parallel to the sagittal plane of the user. The posture device can be oriented any way about the z-axis. For example, the posture device can be moved up or down (such as along the y-axis), or left or right (such as along the x-axis), or rotated about the z-axis.

In one embodiment, in normal usage, a posture device does not have to be in an up/down orientation. For example, the device, a wearable device, could be clipped to a clothing of a user. To illustrate, the device could be a tie clip, and can be normally used in a horizontal position.

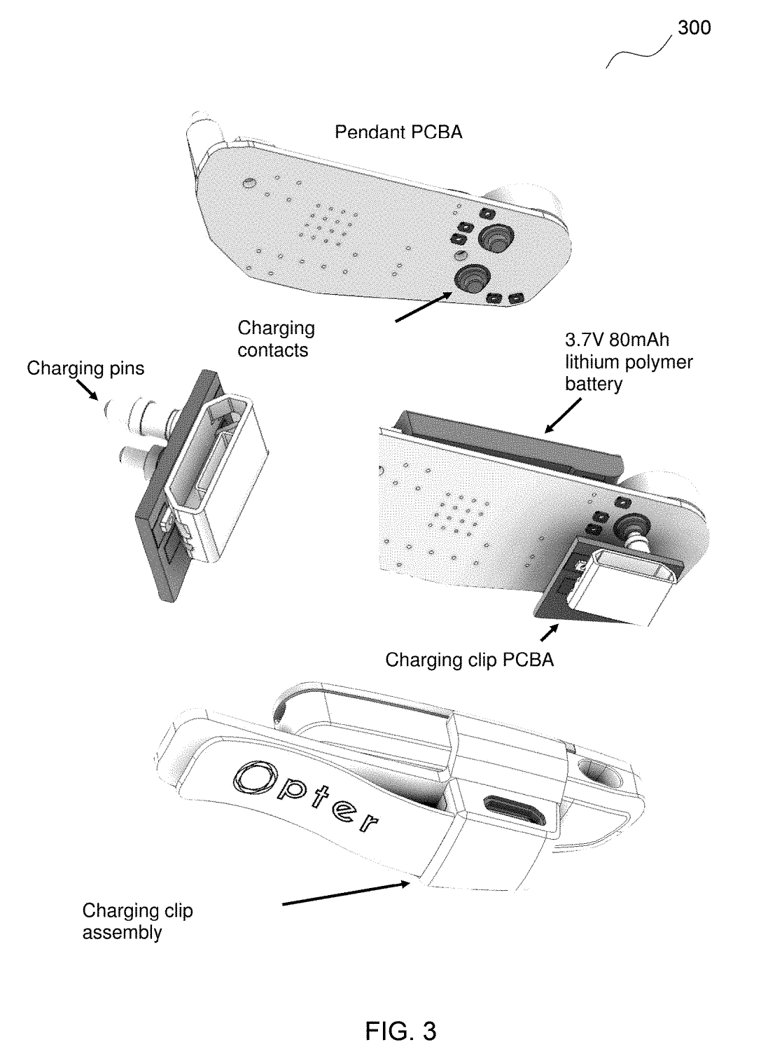

FIG. 3 shows embodiments 300 of a posture device and a clip. In one embodiment, the posture device could include charging pins to charge the posture device. A separate clip could clamp onto notches or indentations on the sides of the posture device. The separate clip could include a charging port, such as a micro-USB connector. When the clip clamps or grabs onto the posture device, charging contacts or pins in the clip would be aligned to the corresponding charging pins at the posture device. Via the charging port at the clip, the posture device could be charged.

Note that in the configurations shown in FIG. 3, the posture device, in operation, may not be in a up/down orientation. The device could be aligned horizontally. In one approach, before using such a device to monitor posture, a user first calibrates the device in the orientation where it is normally used. Then the user starts using the device to measure her postures.

In one approach, posture alerts do not occur if the user has silenced the device by using a specific gesture, such as turning the device upside down. This could save power consumption by the device.

Another power saving approach can depend on turning off the device if the device is detected not to be worn. One approach to determine if the device is not being worn can be based on comparing a range of posture vectors over a period of time, such as 3 seconds. If during the period, the deltas among the vectors are not beyond the noise level of, for example, the accelerometer in the device, the device can be assumed as not being worn, and can go into a sleep mode, with tracking disabled.

In one embodiment, a posture device could be configured to notify the user after the user has a poor posture for a duration of time. In one embodiment, this duration of time could be set by the user. For example, the duration of time can be 30 seconds, 1 minute, 2 minutes, 4 minutes or other durations of time. In one approach, if the user has a good posture during this duration of time, the user would not be considered as having a poor posture. In another approach, if 5% or more of the user's posture is good during this duration of time, the user would not be considered as having a poor posture. This % or other percentages could be set by the device. Reducing the need or frequency to notify the user could extend the battery life of the device.

In one embodiment, since a posture device is configured to fit into the shape of an aesthetically pleasing and stylistic jewelry, such as a pendant, printed circuit board(s) for the posture device could be in different shape. Typically, a printed circuit board applicable would be curved, or have different curvatures to fit into the shape of a corresponding jewelry.

Some jewelries could be relatively small. One embodiment doesn't have to include an external physical button/switch, which could allow the posture device to be smaller; and/or could be easier to make the posture device water resistant or waterproof. And without the restriction of an external button/switch, it could be easier to create an aesthetically-pleasing design for a posture device.

In one embodiment, a posture device could include a vibration motor to alert the user via vibration. In one embodiment, a posture device doesn't include a vibration motor, and could notify the user via a mobile device (e.g. mobile phone) coupled to the posture device. Without the need to vibrate to notify the user would reduce power consumption of the device.

In one embodiment, a posture device can be configured to fit into a wearable device or a jewelry (such as a pendant or a necklace) of the user. Such a posture device can be a posture component that can be, for example, integrated into, inserted in, incorporated into, embedded into, taped to, attached to, secured to, and/or enclosed by a wearable device of the user. In one embodiment, the posture component may not include a vibration motor and/or may not include an external physical switch. The posture component could be wirelessly coupled to the user's mobile device, which could notify and/or alert the user regarding her posture as in the different embodiments described herein.

In one embodiment, a posture device or a lifestyle-monitoring-wearable apparatus can be configured to operate as a pedometer. For example, the accelerometer for posture measurement could also operate as a pedometer.

The categorization of ranges, such as “good,” et al. can use different angle ranges depending upon whether the user is walking or not, as determined by the pedometer.

In one embodiment, the apparatus can be used to determine, for example, if the user is wearing the apparatus, if the user has a restless sleep, if the user is exercising, or if the user has been exercising in a moderate or vigorous manner. The determination can be performed at the apparatus, and can be based on machine learning.

In one embodiment, the machine learning approach does not have to depend on the orientation of the apparatus. In the following, running is used to explain the approach.

As the user is running, the apparatus can capture raw outputs from the accelerometer, such as at 25 Hz. A number of moving averages, such as 4, can be calculated. Each moving average can have a different time scale, such as 1 second, ¼ of a second, and 10 seconds.

With the 1 second time scale as an example, the apparatus could calculate the average of the x, y, and z values of the vector outputs during the previous 1 second. So, the apparatus can get 3 numbers.

Assume that the apparatus uses 4 moving averages. In this example, at 25 Hz, the apparatus could have 12 average numbers, and they can be (x1, y1, z1); (x2, y2, z2); (x3, y3, z3); and (x4, y4, z4). Out of the four sets, one set can be selected as a base line, and is used to subtract from the other three sets. In a general sense, this process can represent removing the gravity pull.

Assume that the apparatus uses (x4, y4, z4) as the base line. After the subtraction, the apparatus could have 9 modified average numbers, and they could be (x1, y1, z1); (x2, y2, z2); and (x3, y3, z3). Then each group of numbers can be combined together. For example, each group of numbers can be added together to form 3 numbers, and they could be (|x1|+|y1|+|z1|), (|x2|+|y2|+|z2|), and (|x3|+|y3|+|z3|), the sum of the absolute values of the components. These numbers could be known as pseudo magnitudes or L1 magnitude.

At 25 Hz, the apparatus could end up with 25 sets of pseudo magnitudes every second.

Then the apparatus can form another average. For example, at every minute, the apparatus generates 3 averages, one for each of the time scales. In other words, the apparatus could find the averages of all the (x1+y1+z1), 25*60 of them, to get a number. The apparatus now could have 3 global averages at every minute.

The next step can be generating a dot product of the 3 global averages with 3 Fisher's Linear Discriminants. The Discriminants can be found by observing large number of running data. With the dot product, the apparatus could end up with a final number. Again based on observing large number of running data, the apparatus could identify thresholds delineating among, for example, not running, running moderately, and running vigorously. Depending on where the final number resides, the apparatus could identify whether the user has been running, or how vigorously the user has been running.

In one embodiment, by using different Fisher's Linear Discriminants, the apparatus can determine other activities the user has been involved in, such as if the user had a restless or restful sleep.

In one embodiment, a lifestyle-monitoring-wearable apparatus could have wood (such as bamboo) on the front side and plastic on the back. Bamboo on the front side or as a front panel made of bamboo could be more aesthetically pleasing than, for example, plastic. The front side can be where the protrusions of the radiation-monitoring module are positioned.

In one embodiment, there can be two charging indentations at the back of the apparatus. The two indentations could go into two pins at a charging station to charge the apparatus. One example is as shown in FIG. 3.

In one approach, the charging station can be directly connected to a wall power outlet for charging the apparatus.

In another embodiment, the charging station can be incorporated into a clip to form a clipping charger. FIG. 3 shows an example. The clipping charger can include two charging pins, an attaching clamp and a charging port, such as a micro-USB charging port. The two charging pins could be positioned to go into the two indentations at the apparatus. The attaching clamp could be positioned to grab onto two corresponding slots on the sides of the apparatus.

With the clamp and the pins, the clipping charger can be firmly attached and aligned to the lifestyle-monitoring-wearable apparatus. Then the clipping charger also could be used to attach the apparatus to a user, such as to the clothing of the user. To illustrate, the clipping charger could clip onto the neck ridge of the user's T-shirt.

In one embodiment, an app on the user's mobile phone (or other type of mobile/computing device) could provide different user interfaces to show monitored measurements, analysis results, and/or ways to improve the user's lifestyle. For example, the app could provide 3D posture visualizations based on measurements by the apparatus.

In another embodiment, there could be a Sunscreen Timer, which can be used to illustrate a countdown till the user-applied sunscreen substantially loses effectiveness. In one approach, while the sunscreen is still considered to be effective, the app may not count any UV that the user is exposed to against the user's accumulated daily exposure.

In one embodiment, the app could provide a Blue Lightbulb to show pictorially if the amount of blue light at the time of measurement can be too bright or dim enough based on the time of day. On the Blue Lightbulb, there could also be a “T sec ago” label showing the time elapsed since the last measurement. Such visualization could increase the confidence level of the user that blue light sensing is working even if the corresponding detector is covered, with the display not updating right away. Similar “T sec ago” label can also be used by a UV Sun display to show pictorially UV exposure to the user.

One embodiment includes an Escalating Alarm Clock feature. This feature could turn the apparatus into an intelligent alarm clock. For example, at the time when the user should get an alert, the apparatus could vibrate gently at first. With no feedback from the user, the vibration could increase in intensity. The escalation could extend to playing sound from the user's corresponding mobile device. The user could stop the alarm by providing feedback, such as opening the app, or moving (as sensed by the apparatus), which could be the user taking a certain number of steps, for instance 7. Via escalation, the chance of waking up or annoying others in the vicinity of the user should be reduced.

A number of embodiments have been described regarding a lifestyle-monitoring-wearable apparatus wirelessly coupled to a mobile device of a user. In one embodiment, the apparatus could be wirelessly coupled to a desk top computer or other types of computing devices.

In different embodiments, the lifestyle-monitoring-wearable apparatus can perform different functions, such as improving posture to reduce back pain and boost mental health; vibrating when the user slouches (the user could be slouching forwards or sideways); notifying the user via phone notification when it's time to put on sunscreen; measuring how harsh the UV is and how long the user has been exposed; getting higher quality sleep by sleeping at the right times and avoiding too much light at night; monitoring when the user sleeps, when the user wakes up, and how much light the user has been getting throughout the day, which could influence the user's circadian rhythm; potentially reducing the user's risk of a swathe of diseases including cancer, diabetes, and heart disease by getting enough exercise and staying active throughout the day; reminding the user to get up and move around if the user has been sitting for too long; and/or tracking the user's steps, weekly exercise, and calories.

The various embodiments, implementations and features of the invention noted above can be combined in various ways or used separately. Those skilled in the art will understand from the description that the invention can be equally applied to or used in other various different settings with respect to various combinations, embodiments, implementations or features provided in the description herein.

The invention can be implemented in software, hardware or a combination of hardware and software. A number of embodiments of the invention can also be embodied as computer readable code on a computer readable medium. The computer readable medium is any data storage device that can store data which can thereafter be read by a computer system. Examples of the computer readable medium include read-only memory, random-access memory, CD-ROMs, magnetic tape, optical data storage devices, and carrier waves. The computer readable medium can also be distributed over network-coupled computer systems so that the computer readable code is stored and executed in a distributed fashion.

Numerous specific details are set forth in order to provide a thorough understanding of the present invention. However, it will become obvious to those skilled in the art that the invention may be practiced without these specific details. The description and representation herein are the common meanings used by those experienced or skilled in the art to most effectively convey the substance of their work to others skilled in the art. In other instances, well-known methods, procedures, components, and circuitry have not been described in detail to avoid unnecessarily obscuring aspects of the present invention.

Also, in this specification, reference to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments mutually exclusive of other embodiments. Further, the order of blocks in process flowcharts or diagrams representing one or more embodiments of the invention do not inherently indicate any particular order nor imply any limitations in the invention.

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

What is claimed is:

1. A computing device comprising:
a display;
a wireless transceiver; and
a processor coupled to the display and the wireless transceiver,
wherein the wireless transceiver is configured to receive data regarding a plurality of sets of values from a wearable posture device of a user, each set regarding three values representing three orthogonal acceleration values from an accelerometer in the wearable posture device, with the three orthogonal acceleration values being used to provide information regarding a posture of the user,
wherein the processor is configured to operate on the received data to generate visualizations tracking the postures of the user, and
wherein the display is configured to present the visualizations generated.

2. A wearable device for a user comprising:
an accelerometer;
a wireless transceiver; and
a processor coupled to the accelerometer and the wireless transceiver, the processor being configured to at least extract, at preset intervals, a plurality of sets of three orthogonal acceleration values from the accelerometer, with each set of the three orthogonal acceleration values being used to provide information regarding the user's posture,
wherein the wireless transceiver is configured to transmit data regarding the plurality of sets of values to a computing device.

3. A wearable device as recited in claim 2, wherein the computing device being a portable computing device that is paired for wireless communication between the wearable device and the portable computing device.

4. A wearable device as recited in claim 3, wherein the portable computing device include an application program operating thereon that processes the transmitted data regarding the plurality of sets of values to the portable computing device.

5. A wearable device as recited in claim 2, wherein the wearable device is, when worn, secured to the head of the user.

6. A wearable device as recited in claim 2, wherein the wearable device is, when worn, secured to about the neck of the user.

7. A wearable device as recited in claim 2, wherein the wearable device comprises a radiation sensor configured to monitor radiation of a range of predetermined wavelengths.

8. A wearable device as recited in claim 7, wherein the range of predetermined wavelengths pertains to blue light.

9. A wearable posture device for a user comprising:
an accelerometer;
an output generator; and
a processor coupled to the accelerometer to
receive data regarding a plurality of sets of values, each set regarding three values representing three orthogonal acceleration values from the accelerometer, with the three orthogonal acceleration values being used to provide information regarding a posture of the user;
operate on the received data to analyze and track the postures of the user; and
notify the user by generating an output via the output generator to alert the user to improve on the user's posture, if the user's posture needs to be improved.

10. A wearable posture device as recited in claim 9, wherein the wearable posture device is configured to be calibrated depending on having the device at a preset position for a predetermined amount of time.
